(19) United States
(12) Patent Application Publication
(10) Pub. No.: US 2023/0366881 A1
(43) Pub. Date: Nov. 16, 2023

(54) IMAGE QUANTIFICATION SYSTEM FOR ESTIMATION OF VIRAL LOAD BASED ON DETECTION BY RAPID ANTIGEN TEST

(71) Applicant: Hai Kang Life Corporation Limited, Shatin (HK)

(72) Inventors: Cheung Hoi Yu, Shatin (HK); Shui Ying Tsang, Shatin (HK); Wing Cheong Chan, Shatin (HK)

(21) Appl. No.: 18/127,887

(22) Filed: Mar. 29, 2023

(30) Foreign Application Priority Data
Mar. 29, 2022 (HK) HK 32022050763.8

(51) Int. Cl. G01N 33/569; G01N 21/78; G01N 33/543; G06T 7/90

(52) U.S. Cl. CPC G01N 33/56983; G01N 21/78; G01N 33/54388; G06T 7/90; G01N 2021/7766

(57) ABSTRACT

The application relates to a method and device for quantitatively analyzing the detection results of lateral flow-type test kits. Disclosed is a method for quantitatively analyzing the test results of a lateral flow-type test kit wherein the test kit includes a result area in which a control C-line region and a detection T-line region are set. The method includes: determining an analyte concentration range that the test kit can detect; diluting a sample into a plurality of sample dilutions having different pre-determined analyte concentrations within the determined analyte concentration range; applying each sample dilution having a pre-determined analyte concentration to a sampling well on the rapid test kit for the analyte, so as to obtain separate resulting images with different color intensities on the T-line region corresponding to different pre-determined analyte concentrations; calculating a corresponding color intensity index values for the T-line regions of each resulting image, respectively; and creating a continuous curve by fitting the corresponding plurality of color intensity index values to the plurality of different pre-determined analyte concentrations, wherein the corresponding analyte concentration can be obtained based on the color intensity index value at any point on the continuous curve.

### TECHNICAL FIELD

The present disclosure relates to the quantitative analysis of lateral flow kits. Specifically, the present disclosure relates to a method and apparatus for quantitative analysis of the test results of a lateral flow-type test kit.

### BACKGROUND

The lateral flow-type kit is a rapid, simple, and practical bioanalytical technique that can be used to detect a variety of biomolecules such as proteins, antibodies, antigens, etc. This kit utilizes the interaction between specific biomolecules to detect the molecule of interest. The principle of the lateral flow kit is based on immunology and paper chromatography techniques, which enable the detection of the target substance by fixing a specific biomolecule on a strip or film, coating the sample at one end of the kit, diffusing along the filter paper or film in the kit, and after contact with the reactant, a visible color or signal is generated. Lateral flow kits have the advantages of simple operation, fast, portable and low cost, so they are widely used in the fields of rapid diagnosis and detection, such as pregnancy tests, influenza virus detection, myocardial infarction detection, hepatitis virus detection, drug testing, agricultural testing, etc.

There are currently many lateral flow test kits on the market for different purposes from different manufacturers, such as human chorionic gonadotropin (HCG) rapid test kit for detecting early pregnancy, hepatitis B virus surface antibody test kit, COVID-19 antigen rapid test kits, etc. Usually, the tester collects different types of body fluid samples (such as urine, blood, nasal cavity or nasopharyngeal swabs, etc.), and then adds the sample mixed with enough test solution into the sample well of the lateral flow test kit, and determines whether the test T-line region on the test kit has developed color to determine whether the test is negative or positive. Compared with the gold standard of similar testing, such as nucleic acid detection, lateral flow detection has the advantages of fast and efficient, low cost, high specificity and sensitivity, wide application, strong stability, no special equipment and reagents, no centralized testing, and intuitive result judgment because of its simple operation process.

The lateral flow test kits have been in use for a long time and there have been many instances of related applications. In the COVID-19 outbreak, for example, COVID-19 rapid antigen tests were widely used as a preliminary screening tool, and because of their simple and easy operation, they became an important means of home self-testing for residents during stringent epidemic prevention and control. The lateral flow kit allows people to self-test at home to achieve the goals of early detection, early isolation and early treatment, and is successfully applied to identify early patients and cut off the virus transmission chain. However, lateral flow kits are generally only used for qualitative analysis of certain diseases, such as for rapid diagnosis and screening, and there are no attempts to extract any quantitative information from them, such as the content of viral proteins in the sample.

Quantitative analysis for self-detection, telemedicine and health records is a revolutionary advancement. By performing self-testing and quantitative analysis at home, it allows doctors to determine, without face-to-face contact, the extent of infection for a patient with disease and to provide more accurate analysis results and recommendations. Quantitative analysis can determine a patient’s health by measuring the concentration of specific biochemicals in blood, urine, or other body fluids. These biochemicals include drugs, metabolites, proteins, etc. Quantitative analysis can help doctors better understand the physiological state of patients, better understand and observe the health status of patients, and adjust treatment plans as needed.

Taking FIG. 1 as an example, the antigen concentration (viral load) of COVID-19 patients is constantly changing throughout the course of the disease (infection cycle, about 12 days). If the approximate concentration of antigens that indicates viral load in the body can be monitored for several days, combined with the patient’s clinical symptoms and personal information, the stage of the patient’s disease course can be inferred. Medical workers can then give professional medical guidance and nursing advice according to disease stage. The patient or his or her caregiver can also use this to predict likely trends in the coming period.

However, at this stage there is a lack of convenient and effective monitoring tools to enable medical workers or patients to obtain quantitative antigen concentrations in a timely and continuous manner to obtain quantitative antigen concentrations. As the only and commonly used detection tool to determine whether the virus is present in the body of people in home isolation, antigen rapid test kits can only provide information on whether they are infected with the virus or not, and cannot provide objective and quantitative reference values on the current viral load for patients in home isolation or their caregivers to allow patients or their caregivers to understand their disease progression. Although viral load testing can be done to inform patients about the condition, it is not suitable for all patients in terms of time and economic cost. Therefore, patients infected with COVID-19, whether they are severely ill in hospitalization or with mild to moderate disease at home, need convenient and fast monitoring tools to enable healthcare workers or the patients themselves to measure the viral load so as to understand the progression of the disease.

Further, because the current market has a wide range of lateral flow test kits for different uses from different manufacturers, the quality and stability of the kits often do not provide reliable and accurate test results. The kit manufacturers also lack quality control tools for the kit to improve the reliability and accuracy of the test results. By using quantitative techniques, we can perform quality control on the kits, ensuring their accuracy and reliability. This can be achieved by preparing a series of standard curves containing substances of known concentration. We can use these standard curves for quality control to ensure that the kit accurately measures the amount of substance in the sample. This quality control guarantees consistent performance and accuracy of the kit, increasing the reliability and accuracy of the test.

The present disclosure uses artificial intelligence and image processing technology to detect the result area of the kit and reduce the impact of the environment on the result quantitation, so that the quantitative technology can be implemented without the help of professionals and instruments. Existing quantitative techniques, such as the U.S. Pat. application US20210295505A1, discloses an automated image analysis comprising colorimetric determination of analyte concentration platform for rapid antigen detection to quantify proteins, but the quantification process still relies on a laboratory environment and cannot be used at home. After microwave heating to conduct the colorimetric microwave-accelerated bioassay MAB procedures, the user prepares the colorimetric assay in a microplate and color develops in each microwell. After this color development and the reaction is complete, the user can use a remote image capture device to capture a digital image of the 250-well microplate. After capturing this digital image, the user can transmit the digital image to the analyte detection and analysis computer for image processing, for example via a wide area network. The digital image analysis computer calculates the pixel intensity in each well containing the colorimetric assay and outputs the analyte concentration in each well based on the calculated pixel intensity.

US20210295505A1 involves the enzyme-linked immunosorbent assay (ELISA). This is a technique widely used in the field of bioassays to quantify proteins. In the method disclosed in US20210295505A1, the microplate is used as a support for the antibody to fix the antibody in each well, and the microplate has wells with standard size in a specific arrangement. In addition, the analyte reacts in a stationary solution, and color development in the microwells comes from the reaction of the enzymes in solution that detect antibody conjugation with its substrate. The pixel intensity of color rendering in each well can be measured using a plate reading device at a given wavelength.

However, the quantitative analysis technique for this enzyme-linked immunosorbent assay (ELISA) is not suitable for lateral flow kits. Unlike the US20210295505A1 microplates with standard-sized wells with a specific arrangement of fixed antibodies, lateral flow kits typically use lateral flow rapid assay technology. This technique typically uses nitrocellulose membranes to capture the molecule of interest. There is no standardized size for kits from different manufacturers. The size of the nitrocellulose membrane that hosts the sample solution and the result window used for observation vary. Moreover, lateral flow technology is used so that the solution mixed with the sample is not stationary, but must flow along the length of the nitrocellulose membrane. In addition, the lateral flow-type kit develops color on the T-line region from gold or other stains captured on nitrocellulose membranes to detect antibody conjugation. Since there is no design standard for the color intensity range for color development on the T-line region of the lateral flow antigen or antibody rapid test kits manufactured by different manufacturers, the test is usually only based on whether the detection T-line region on the test kit has color development to judge whether virus is present or not. The color intensity after color development is not measured, and there is no special equipment for measuring the intensity. Moreover, the quantitative analysis of ELISA must be carried out in large testing centers, which is very inconvenient and time-consuming.

### INVENTION CONTENT

The inventor of the present application found that, although the detection T-line region of the lateral flow kit from different manufacturers has different color development ranges for test solution samples having different virus concentrations, the color intensity of the T-line region is positively correlated with the antigen or antibody concentration, Moreover, the color intensity distribution curves of the detection T-line region of the lateral flow kits from different manufacturers are basically the same relative to the virus concentration. In view of this, the inventor of the present application proposes a method for quantifying the detection results of a lateral flow-type kit. Specifically, the inventors propose that the color intensity distribution curves of the color intensity of the detection T-line region of the lateral flow-type kit of different brands can be obtained relative to the concentration of the detected analyte. The individual color intensity index values on this color intensity distribution curve are related to the color intensity of the color developed and reflect the corresponding analyte concentration. Therefore, analyte concentrations corresponding to any color intensity index value on the distribution curve can be obtained from this color intensity distribution curve.

Based on the above inventive ideas, embodiments of the present disclosure provide a method for quantitative analysis of the detection results of the lateral flow-type kit and a computer device to quantitatively analyze the detection results of the lateral flow type kit. The method disclosed in the present application is further improvement of lateral flow-type kits that have been widely used for many years in the medical and pharmaceutical fields. The present application uses computer technology to break through the limitation of kits that can traditionally only be analyzed qualitatively, and through accurate calculation and quantitative processing, it now has more accurate, more efficient and convenient detection capabilities. This revolutionary use not only greatly improves the efficiency and convenience of testing in the medical and pharmaceutical fields, but also provides a powerful quality control tool for kit manufacturers.

According to the first aspect of the present disclosure, there is provided a method for quantitative analysis of the detection results of the lateral flow rapid test kit, wherein the test kit comprises a result area with a quality control C-line region and a detection T-line region, the method comprising: determining an analyte concentration range that the test kit can detect; diluting a sample into a plurality of sample dilutions having different pre-determined analyte concentrations within the determined analyte concentration range; applying each sample dilution having a pre-determined analyte concentration to a sampling well on the rapid test kit for the analyte, so as to obtain separate resulting images with different color intensities on the T-line region corresponding to different pre-determined analyte concentrations; calculating a corresponding color intensity index values for the T-line regions of each resulting image, respectively; and creating a continuous curve by fitting the corresponding plurality of color intensity index values to the plurality of different pre-determined analyte concentrations, wherein the corresponding analyte concentration can be obtained based on the color intensity index value at any point on the continuous curve.

According to the second aspect of the present disclosure, a computer device is provided, comprising: a database storing the continuous curve representing color intensity index values associated with analyte concentrations obtained according to the method of claim 1; a processor; and a memory unit having stored thereon a computer program which, when executed on the processor, causes the processor to perform the steps of: acquiring an image of a lateral flow test kit with the test result; locating the T-line region in the image of the kit; analyzing the image to determine a color intensity index value for the T-line region; and obtaining the analyte concentration value corresponding to the color intensity index value based on the continuous curve.

According to a third aspect of the present disclosure, there is provided a computer-readable storage medium, wherein a computer program is stored. When the computer program is run on a computing device, the computing device performs a step of quantitative analysis of the detection results of the lateral flow type kit according to the foregoing implementation.

The present invention provides the use of AI technology to easily and quickly quantify and record viral load, so that patients or caregivers can understand the stage of disease progression, and at the same time can store and track viral load changes over time, making reasonable predictions about the future development of the disease. The present invention can be applied to all lateral flow test kits, adding new functions on top of the basic application of the rapid kit that has been widely used in large quantities. The new functions are not limited by time, space and operator technology, improving the convenience of use, expanding the use scenarios, and making up for the lack of monitoring tools on the market for patients in home isolation with mild and moderate diseases, thereby reducing labor and economic costs at the social level. Moreover, for hospitalized critically ill patients, compared with frequent nucleic acid testing, the present invention can also provide timely analysis of relative viral load, to achieve continuous monitoring and reduce the pressure of hospital overload. The method of quantitative analysis of the test results of the lateral flow kit disclosed in the present application provides the user of the kit with a tool to quickly and conveniently obtain viral load at home or in a medical institution lacking a testing center to understand the course of the disease, and also provides a quality control tool for lateral flow test kit manufacturers.

According to the embodiments described below, these and other aspects of the present disclosure will be clear and will be clarified with reference to the embodiments described below.

### DESCRIPTION OF THE DRAWINGS

In the description of exemplary embodiments in conjunction with the accompanying drawings below, further details, features and advantages of the present disclosure are disclosed, in the drawings:

FIG. 1 schematically shows the change in antigen concentration (viral load) throughout the infection cycle;

FIG. 2A schematically shows the correlation between antigen concentration and the color intensity index of the T-line region color rendering of the first antigen rapid test (ART) kit;

FIG. 2B schematically shows the correlation between antigen concentration and the color intensity index of color rendering in the T-line region of the second antigen rapid test (ART) kit;

FIG. 3 schematically illustrates a flowchart 300 of a method for quantitative analysis of the test results of the lateral flow antigen rapid test (ART) kit;

FIG. 4A schematically illustrates the structure of the ART kit 400;

FIG. 4B schematically shows rotating the ART kit 400 image of FIG. 4A into a landscape position;

FIG. 5 schematically illustrates a flow chart 500 for quantitative analysis of the test results of a lateral flow-type kit performed by a computer device according to an embodiment of the present application;

FIG. 6 schematically shows an algorithm flowchart 600 for locating the T-line region in the image of a lateral flow-type kit according to an embodiment of the present application;

FIG. 7 schematically illustrates a block diagram of a computing device according to an embodiment of the present application.

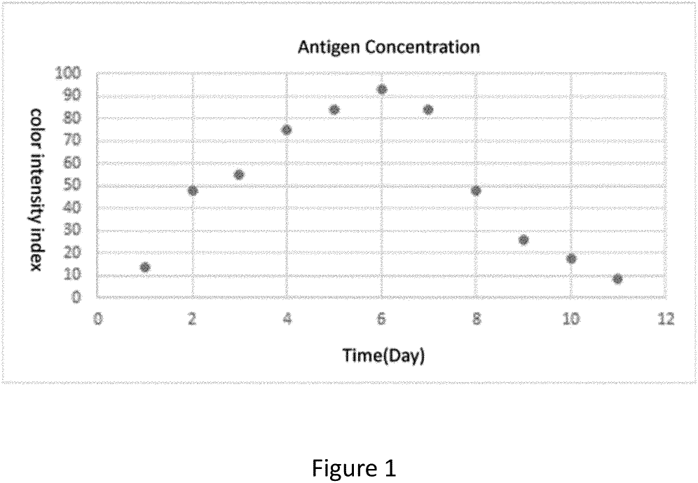

### SPECIFIC EMBODIMENT

The embodiment of the present disclosure provides a scheme involving a lateral flow rapid test quantitative analysis technique, specifically illustrated by the following COVID-19 rapid antigen test as an exemplary embodiment. Those skilled in the art will understand that the methods of embodiments of the present disclosure may be applied to other lateral flow test kits.

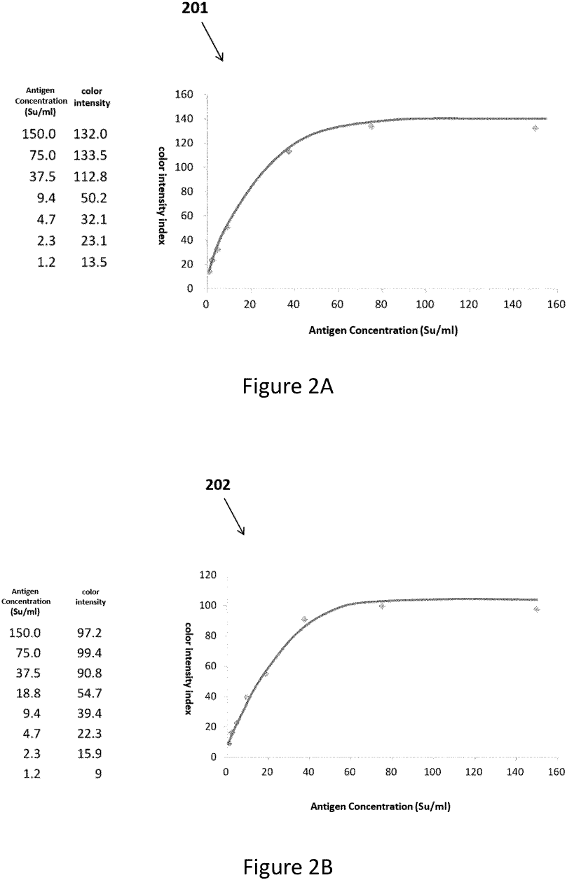

FIG. 2A and FIG. 2B respectively show for a plurality of pre-determined antigen concentrations of virus dilution, the corresponding color intensity index for the color rendering of the T-line region for two different brands of COVID-19 rapid antigen kit. FIG. 2A shows the test data and analysis for the first rapid antigen kit. The first kit has a color intensity index ranging from 0-160 for T-line region color rendering. For antigen concentration range 0-160Su/ml, the pre-determined antigen concentration in this concentration range is 1.2Su/ml, 2.3Su/ml, 4.7Su/ml, 9.4Su/ml, 37.5Su/ml, 75.0Su/ml, 150Su/ml. These were applied to the sampling wells of the rapid antigen test kit. After completion of the reaction and color development, the corresponding color intensity indices in the T-line area are 13.5, 23.1, 32.1, 50.2, 112.8, 133.5, 132.0, respectively. Graph 201 of FIG. 2A shows a continuous curve of the color intensity index relative to antigen concentration (Su/ml) obtained based on the above data. FIG. 2B shows the second rapid antigen kit test data and analysis. The color intensity index of the T-line region of the second kit ranges from 0-120, and the pre-determined antigen concentrations in this concentration range are 1.2Su/ml, 2.3Su/ml, 4.7Su/ml, 9.4Su/ml, 18.8Su/ml, 37.5Su/ml, 75.0Su/ml, 150 Su/ml. These were applied to the sampling wells of the rapid antigen kit, and the corresponding color intensity indices for color development in the T-line region after the reaction and color development were completed are 15.9, 22.3, 39.4, 54.7, 70.8, 99.4, 97.2. Graph 202 of FIG. 2B shows a continuous curve of the color intensity index relative to antigen concentration (Su/ml) obtained based on the above data. From the above test data shown in FIGS. 2A and 2B and graphs 201, 202, it can be seen that in the two antigen rapid tests. The range of color intensity indices for color rendering in the T-line region of the ART kit is different, and the color intensity index of color rendering is different for virus dilutions of the same predetermined antigen concentration. However, the shape of the color intensity index relative to the antigen concentration (Su/ml) in graphs 201 and 202 is essentially the same.

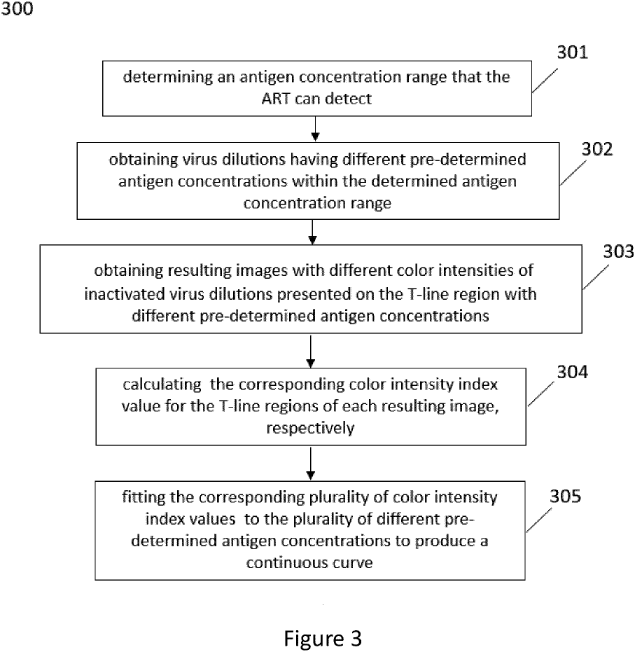

FIG. 3 schematically illustrates a flow chart 300 of a method for quantitative analysis of the results of the lateral flow antigen rapid test (ART) kit.

First, in step 301, it is necessary to determine the antigen concentration range that the antigen rapid test (ART) kit can detect. Different types of ART kits can detect different ranges of antigen concentrations. Taking the widely used COVID-19 rapid antigen kit as an example, the antigen concentration that the ART kit can detect is roughly 0-150 Su/ml. Those skilled in the art can understand that COVID-19 rapid antigen test kits from different manufacturers can also detect different ranges of antigen concentrations.

After determining the antigen concentration range that the specific ART kit can detect, in step 302, the virus sample is diluted to obtain a plurality of different pre-determined antigen concentrations within the determined antigen concentration range. For example, the antigen concentrations listed in FIG. 2A can be obtained by diluting the virus to 1.2 Su/ml, 2.3 Su/ml, and 4.7 Su/ml 9.4Su/ml, 37.5Su/ml, 75.0Su/ml, 150Su/ml. For safety reasons, inactivated viruses can be used here.

Different pre-determined antigen concentrations of inactivated virus dilutions are applied to the sampling wells on the antigen rapid test kit, according to the specifications of the specific antigen rapid test kit. After the reaction is completed and the color development is stable, in step 303, obtain respective result images with different color intensities rendered on T-line regions for inactivated virus dilutions with different antigen concentrations. Different image capture devices can be employed to acquire images of the T-line region, for example, cameras or scanners coupled to a fixed terminal or mobile communication device.

Input the result images into the computing device. The resulting image can be processed as usual in the field of image processing, such as converting a color map to a grayscale map to extract information in the grayscale image that reflects the color intensity. In step 304, according to the color intensity information extracted for inactivated virus dilutions having different antigen concentrations, the corresponding plurality of color intensity index values for the T-line region of each result image is separately calculated. For example, for the antigen concentration listed in FIG. 2A above, the corresponding color intensity index values of the first kit in the T-line region are 13.5, 23.1, 32.1, 50.2, 112.8, 133.5, 132.0, respectively. In step 304, the corresponding plurality of color intensity index values may be fitted to a continuous curve relative to a plurality of different pre-determined antigen concentrations. Because the individual color intensity index values on the continuous curve are related to the color intensity, they reflect the antigen concentration level. Thus, the corresponding antigen concentration value can be obtained based on the color intensity index value at any point on this continuous curve.

In one embodiment, the resulting continuous curve may be segmented. For example, in the first kit example shown in FIG. 2A, the continuous curve may be divided into five segments, according to the order of antigen concentration from low to high, and each segment color intensity index value range corresponds to “very low”, “low”, “medium”, “high”, “very high”. As previously described, the color intensity index range of color rendering in the T-line region of antigen rapid test ART kits from different manufacturers is different, and the color intensity index of color rendering is different for virus dilutions of the same pre-determined antigen concentration. However, since the correlation of the color intensity index of the T-line region of the antigen rapid test ART kit from different manufacturers is similar relative to the antigen concentration (Su/ml), the shape of the resulting continuous curve is basically the same. Thus, for different ART kits, the above five ranges of the continuous curve can correspond to the color intensity index ranges for different ART kits.

In one embodiment, for different ART Kit, for each segment color intensity index value range corresponding to the antigen concentration range of “very low”, “low”, “medium”, “high”, “very high”, respectively, extract the respective intermediate value from color intensity index values of each segment, and make a color patch of the corresponding color intensity index value. For example, five patches with their respective median values may be printed on paper with the corresponding words “Very Low”, “Low”, “Medium”, “High”, and “Very High”, such as on an ART kit or in the accompanying instructions. This allows the user with the test results to compare the T-line region of the ART kit color to the five color patches, which gives an idea of the approximate level of viral load. In another example, the color patch may also be displayed on the display, for example, through an application associated with the ART kit, or a linked web page presented to the user’s computing device display or mobile terminal display, for convenient user comparison.

In another embodiment, in order to provide the user with a more accurate antibody concentration, the obtained continuous curve related to the antigen concentration may be stored in the memory of the computing device, for example, stored in a remote computing device. After obtaining the test results using the ART kit, the user can use an image acquisition device, such as a camera or scanner of a mobile device, to obtain an image of the T-line region with the test result. The image of the T-line area with the test result is then transmitted over the network to a remote computing device. The computing device analyzes the image of the obtained T-line region to determine the color intensity index value of the T-line region, obtains the corresponding antigen concentration according to the stored continuous curve, and transmits the obtained antigen concentration to the user through the network, for example, to the user’s mobile communication device or to the user by e-mail or SMS. This embodiment will be described in detail hereinafter based on a computer device.

In order to obtain a more accurate antigen concentration value, specific continuous curves for different brands or different kit types may be acquired from a plurality of manufacturers of different brands or different kit types.

First, the color intensity range of the test results on the T-line region of the antigen rapid test ART kit needs to be determined for a plurality of different brands or kit types. Then, for each brand or type of antigen rapid test ART kit, the result images with different color intensities presented by inactivated virus dilutions with different pre-determined antigen concentrations on the T-line region were obtained, and the corresponding color intensity index values of the T-line region of each result image were calculated. Finally, for each brand or type of antigen rapid test ART kit, the corresponding multiple color intensity index values are fitted to each brand or type-specific continuous curve relative to a number of different pre-determined antigen concentrations.

In another embodiment, the similarity of continuous curves of different brands or kit types may be used, and only the color intensity range of the test results on the T-line region for a different brand or type of antigen rapid test ART kit needs to be determined to allow the obtained continuous curve to be positioned within a defined brand-specific or type-specific color intensity range. In this embodiment, after determining the color intensity range of the test results on the T-line region of a plurality of antigen rapid test ART kits of different brands or types, there is no need to obtain the corresponding color intensity index values of inactivated virus dilutions of different pre-determined antigen concentrations on the T-line region for antigen rapid test ART of each brand or type. Only the color intensity range determined by the kit needs to be used in a coordinate system with antigen concentration as abscissa and color intensity as ordinate, the start and end points of the previously obtained continuous curve on the ordinate can be determined to obtain the antigen concentration corresponding to a specific brand or type based on the color intensity index value at any point on the continuous curve.

The present application also provides a computer device to achieve quantitative analysis of lateral flow-type antigen or antibody rapid test kit results to obtain an accurate antigen / antibody concentration value of the exemplary embodiment. As mentioned earlier, a dedicated computer device can be set up to store the obtained continuous curve related to antigen concentration in the database of the computing system, and after receiving an image of the T-line region with the test result provided by the user, a more accurate antibody concentration value can be provided to the user based on the image of the T-line region with the test result provided by the user. The computer device may remotely receive images of the ART kit T-line region with test results taken by the user with his own camera or scanner, such as a camera on a mobile communication device. Manufacturers of lateral flow antigen or antibody rapid test kits may also be able to use the computer device of the present application to perform quality control of the manufactured kits.

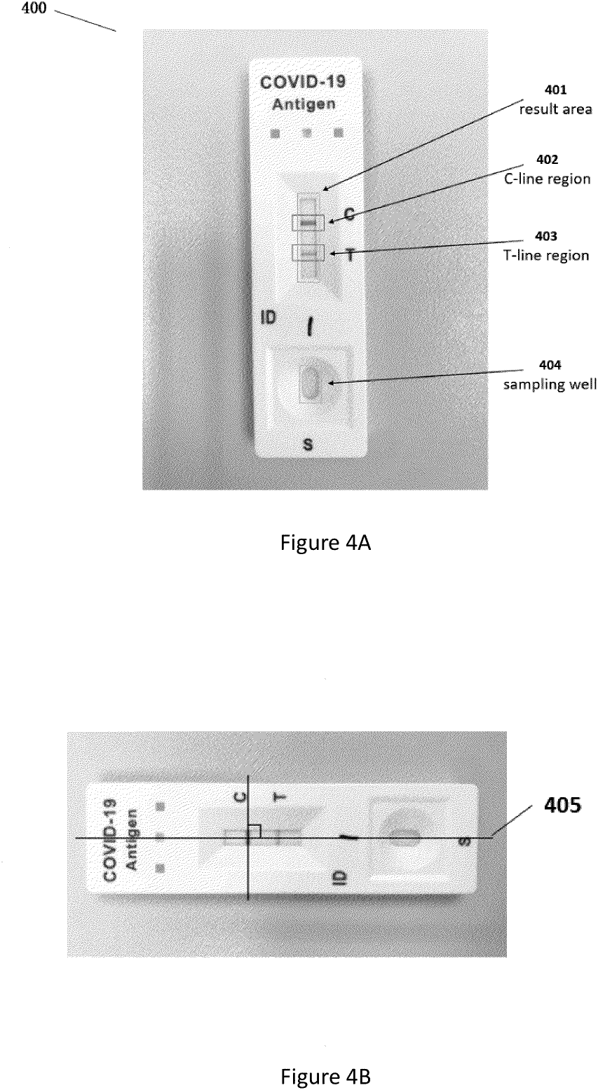

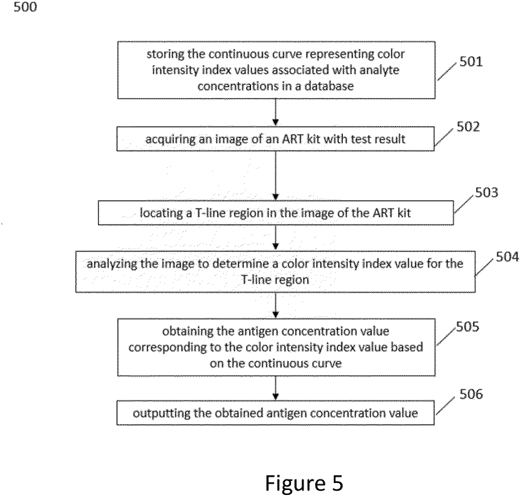

In one embodiment, the kit template may be created separately for different brands or types of antigen or antibody rapid test kits and stored in the memory unit, wherein the kit template comprises a specific continuous curve associated with the antigen or antibody concentration-indicating color intensity index values of the brand or kit type of an antigen or antibody rapid test kit; image of the antigen or antibody rapid test kit; dimensions of individual parts of the kit; and the relative position of each part. For example, FIG. 4A shows a schematic of the COVID-19 Rapid Antigen Test Kit. The rapid antigen test kit includes a result area 401 and a sampling well 404, wherein, the result area includes a control region, i.e., the C-line region 402, and a detection region, i.e., a T-line region 403. For COVID-19 rapid antigen kits from different manufacturers, the size and relative position of C-line region 402 and T-line region 403, are variable. The stored kit template can be utilized to locate the T-line region, which can be found, for example, using the XY offset of the T-line region relative to the edge of the ART kit T-line region. In one embodiment of the present application, the steps of the flow chart 500 shown in FIG. 5 may be implemented by a computer device to quantitatively analyze the detection results of the lateral flow-type antigen rapid test ART kit. In step 501, the antigen concentration associated with a continuous curve representing the color intensity index value is stored in the database; At step 502, acquire an image of the antigen rapid test ART kit with detection results; At step 503, locate the T-line region in the image of the ART kit; At step 504, analyze the image of the ART kit to determine the color intensity index value of the T-line region; and obtain the antigen concentration value corresponding to the color intensity index value according to the continuous curve.

In step 502, after receiving the user-sent image of the ART kit T-line region having detection results, the computer device first extracts the features of the ART kit from its appearance, including the text and patterns on the surface, which is used as a screening standard to automatically identify the kit in the image submitted by the user, and isolate the layout information of the kit to be analyzed from the image.

After extracting the layout information of the ART kit to be analyzed, in step 503, the detection T-line region 403 needs to be located in order to determine the color intensity index value of the T-line region 403. Due to the position of the detection area 401 and the relative position of the C-line region 402 and the T-line region 403 is variable relative to the ART kit of different brands or types of ART kits from different manufacturers, how to locate the position of the T-line region 403 in the ART kit image is the most challenging part. In particular, location of the T-line region 403 in ART kit images that do not have stored kit templates.

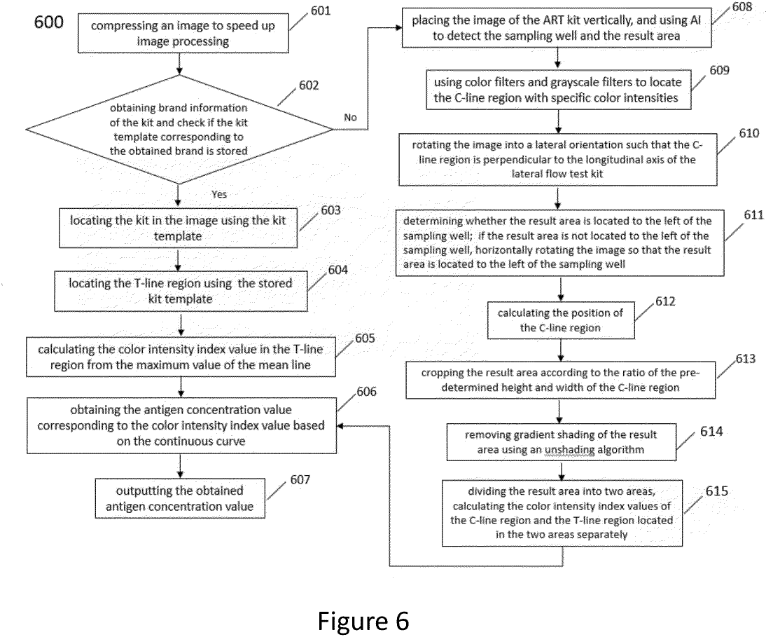

FIG. 6 schematically illustrates an algorithm flow diagram 600 for locating the T-line region according to an embodiment of the present application. First, in step 601, the image is compressed to speed up image processing. In step 602, the computer device of the present application may automatically identify the user submitted information about the kit to be tested. For example, the pattern information extracted from the detection area can be compared with a stored reference pattern for brand or kit-type matching. If the kit template corresponding to the brand or kit type obtained is stored, then in step 603, the stored kit template may be used to identify the kit in the image, followed by step 604, further positioning the T-line region. In one embodiment, the T-line region may be located using the XY offset of the T-line region 403 relative to the edge of the ART kit included in the kit template. After locating the T-line region 403, at step 605, the image of the ART kit is analyzed to determine the color intensity index value for the T-line region. Then in step 606, according to the stored continuous curve to obtain the antigen concentration value corresponding to the color intensity index value. Finally, in step 607, the obtained antigen concentration value is transmitted to the user’s computing device via the network.

If there is no kit template stored in the memory unit corresponding to the brand or type obtained, then the T-line region 403 can be positioned by first locating the C-line region 402, and then taking the C-line region 402 as a reference. In one embodiment, in step 608, the image of the rapid antigen test ART kit is placed vertically, and artificial intelligence AI is used to detect the sampling well 404 and the result area 401, wherein the result area 401 includes a C-line region 402 and a T-line region 403. In one embodiment, at step 609, for example, a color filter and a grayscale filter may be used to locate the C-line region 402 with a specific color intensity. In an alternative embodiment, the C-line region 402 may be located through steps 610-612. At step 610, as shown in FIG. 4B, the image may be rotated into a landscape orientation such that the C-line region 402 is perpendicular to the longitudinal axis 405 of the antigen rapid test ART kit. In step 611, it is determined whether the result area 401 is located on the left side of the sampling well 404; if the result area 401 is not located on the left side of the sampling well, the image is horizontally rotated so that the result area 401 is located on the left side of the sampling well 404. At step 612, the location of the C-line region 402 is calculated. In step 613, the result area 401 is cropped according to the pre-determined ratio of height and width of the C-line region 402. At step 614, the gradient shading of the result area 401 is removed using an unshading algorithm. In step 615, the result area 401 is divided into two areas down the middle, and the color intensity index values of the C-line region and the T-line region are calculated, wherein the C-line region 402 and the T-line region 403 are separately located in the two divided areas. In one embodiment of the present application, the manufacturer of the lateral flow test kit can use the computer device in this patent application to perform quality control on the produced kit. For example, according to the method disclosed above in this application, a standard curve can be established for each kit type, and the standard curve shows the proper correlation between the color intensity and the load of the biomolecules to be tested. For each batch of kits, it is necessary to obtain the batch curve between the color intensity of the batch and the load of the biomolecule to be tested according to the method disclosed above in this application, and compare it with the previously established corresponding standard curve. If the difference between the batch curve and the standard curve is within the specified threshold range, the quality of this batch of kit is considered acceptable; otherwise, this batch of kit will be judged as defective.

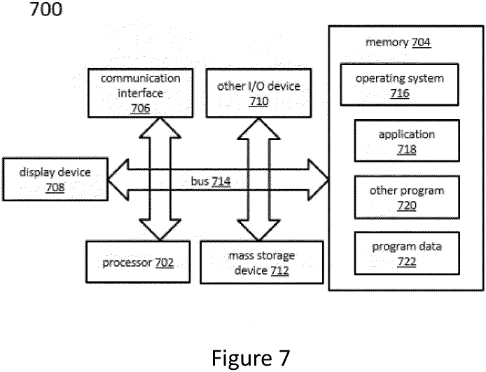

FIG. 7 schematically illustrates a block diagram of a computing device 700 according to an embodiment of the present disclosure. The computing device 700 is used to automate the quantification of test results for a lateral flow-type test kit.

The computing device 700 may be a variety of different types of devices, such as a server computer, a device associated with a client (e.g., a client device), a system on a chip, and/or any other suitable computing device or computing system.

The computing device 700 may include at least one processor 702, memory 704, (at least two) communication interfaces 706, display devices 708, other input/output (I/O) devices 710, and one or more mass storage devices 712 that are capable of communicating with each other, such as via a system bus 714 or other suitable means.

The processor 702 may be a single processing unit or at least two processing units, all of which may include a single or at least two computing units or at least two cores. The processor 702 may be implemented as one or more microprocessors, microcomputers, microcontrollers, digital signal processors, central processing units, state machines, logic circuits, and/or any device that manipulates signals based on operational instructions. Among other capabilities, the processor 702 may be configured to obtain and execute computer-readable instructions stored in the memory 704, mass storage device 712, or other computer-readable medium, such as program code for the operating system 716, program code for the application 718, program code for the other programs 720, etc., to implement the embodiments of the present disclosure that provide for automatic implementation of the method steps for quantitative analysis of assay results for a lateral flow-type test kit.

Memory 704 and mass storage device 712 are examples of computer storage media for storing instructions and data base data that are executed by processor 702 to perform various functions previously described. By way of example, memory 704 may generally include both volatile memory and non-volatile memory (e.g., RAM, ROM, etc.). In addition, mass storage devices 712 may generally include hard disk drives, solid state drives, removable media (both external and removable drives), memory cards, flash memory, floppy disks, optical disks (e.g., CDs, DVDs), storage arrays, network storage, storage area networks, and the like. Both memory 704 and mass storage device 712 may be referred to collectively herein as memory or computer storage media, and may be non-transitory media capable of storing computer-readable, processor-executable program instructions as computer program code that can be used by processor 702 as a specific machine configured to implement the operations and functions described in the examples herein execute.

At least two program modules may be stored on mass storage device 712. These programs include an operating system 716, one or more applications 718, other programs 920, and program data 722, and they may be loaded into memory 704 for execution. Examples of such applications or program modules may include, for example, computer program logic (e.g., computer program code or instructions) for implementing the following components/functions. Although illustrated in FIG. 7 as being stored in memory 704 of computing device 700, modules 716, 718, 720, and 722, or portions thereof, may be implemented using any form of computer-readable medium accessible by computing device 700. As used herein, “computer-readable medium” may include one or more types of computer-readable media, and may include, for example, computer storage media and/or communication media.

Computer storage media includes volatile and non-volatile, removable and non-removable media implemented by any method or technique for storing information, such as computer readable instructions, data structures, program modules, or other data. Computer storage media include, without limitation, RAM, ROM, EEPROM, flash memory, or other memory technology, CD-ROM, digital versatile disk (DVD), or other optical storage device, magnetic cartridge, magnetic tape, disk storage device, or other magnetic storage device, or any other non-transmission medium that can be used to store information for access by a computing device.

In contrast, a communication medium may specifically implement computer-readable instructions, data structures, program modules, or other data in a tuned data signal such as a carrier wave or other transmission mechanism. Computer storage media as defined herein does not include communication media.

Computing device 700 may also include one or more communication interfaces 706 for exchanging data with other devices, such as via a network, direct connection, and the like. The communication interface 706 may facilitate communication within a variety of networks and protocol types, including wired networks (e.g., LAN, cable, etc.) and wireless networks (e.g., WLAN, cellular, satellite, etc.), the Internet, and the like. Communication interface 706 may also provide communication with external storage devices (not shown) in, for example, storage arrays, network storage, storage area networks, and the like.

In some examples, a display device 708, such as a monitor, may be included for displaying information and images. Other I/O devices 710 may be devices that receive various inputs from the user and provide various outputs to the user, and may include a camera, keyboard, remote control, mouse, printer, audio input/output device, and the like.

It will be clear to those skilled in the art that for ease and brevity of description, the specific processes of operation of the systems, apparatus devices, and modules described above may be referred to the corresponding processes in the preceding method embodiments and will not be repeated herein.

By studying the accompanying drawings, the disclosure, and the appended claims, a person skilled in the art can understand and realize variations of the disclosed embodiments when practicing the claimed protected subject matter. In the claims, the words “A and/or B” refer to A, B, or A and B, the word “includes” does not exclude other elements or steps, and the indefinite article “one” or The words “first”, “second”, “third”, “fourth” are used only to distinguish between The words “first,” “second,” “third,” and “fourth” are used only to distinguish elements or steps and do not indicate the order of the elements or steps. The mere fact that certain measures are documented in mutually distinct dependent claims does not indicate that the combination of these measures cannot be used to benefit.

1. A method for quantitatively analyzing a test result of a lateral flow-type test kit, wherein the test kit comprises a result area with a quality control C-line region and a detection T-line region, the method comprising:
determining an analyte concentration range that the test kit can detect;
diluting a sample into a plurality of sample dilutions having different pre-determined analyte concentrations within the determined analyte concentration range;
applying each sample dilution having a pre-determined analyte concentration to a sampling well on the rapid test kit for the analyte, so as to obtain separate resulting images with different color intensities on the T-line region corresponding to different pre-determined analyte concentrations;
calculating a corresponding color intensity index values for the T-line regions of each resulting image, respectively; and
creating a continuous curve by fitting the corresponding plurality of color intensity index values to the plurality of different pre-determined analyte concentrations, wherein the corresponding analyte concentration can be obtained based on the color intensity index value at any point on the continuous curve.

2. The method according to claim 1, wherein the analyte is any chemical substance, which may include drugs, metabolites, proteins, and the like.

3. The method according to claim 1, wherein the sample is an inactivated virus.

4. The method according to claim 1, further comprising: segmenting the continuous curve, each segment having a range of color intensity index values corresponding to “very low”, “low”, “medium”, “high”, ″very high″ sample concentration ranges, respectively.

5. The method according to claim 4, further comprising: making at least one color-patch corresponding to one of the ranges of color intensity index values for the continuous curve segments.

6. The method according to claim 5, wherein the color intensity of the at least one color-patch is the median value of the segment corresponding to the range of color intensity index values.

7. The method according to claim 6, further comprising: making at least one color-block includes printing the color-block on paper or displaying the color-block on a display.

8. The method according to claim 1, further comprising:
storing the continuous curve associated with analyte concentration in a memory of a computer device;
obtaining an image of the T-line region with the test result by an image acquisition device;
analyzing the image of the T-line region to determine the color intensity index value of the T-line region, and obtaining the corresponding analyte concentration according to the continuous curve.

9. The method according to claim 1, further comprising: determining the color intensity range of the test results on the T-line region of a plurality of different kinds of lateral flow test kits.

10. The method according to claim 9, further comprising:
obtaining, for each type of lateral flow test kit, respectively, the result image with different color intensities presented by the different pre-determined analyte concentrations on the T-line area, and calculating, respectively, a corresponding color intensity for the T-line region of each result image; and
for each type of kit, creating a type-specific continuous curve by fitting the corresponding plurality of color intensity index values to the plurality of different pre-determined analyte concentrations.

11. The method of claim 9, further comprising:
for each type of kit, determining, in a coordinate system with the analyte concentration as the abscissa and the color intensity as the ordinate, a starting point and an end point of the fitted continuous curve within the determined color intensity range, whereby the corresponding analyte concentration for the corresponding type of kit can be obtained based on the color intensity index value at any point on the continuous curve.

12. A computer device comprising:
a database storing the continuous curve representing color intensity index values associated with analyte concentrations obtained according to the method of claim 1;
a processor; and
a memory unit having stored thereon a computer program which, when executed on the processor, causes the processor to perform the steps of:
acquiring an image of a lateral flow test kit with the test result;
locating the T-line region in the image of the kit;
analyzing the image to determine a color intensity index value for the T-line region; and
obtaining the analyte concentration value corresponding to the color intensity index value based on the continuous curve.

13. The computer device of claim 12, the computer program, when executed on the processor, further causing the processor to perform the steps of:
creating the respective kit templates for a plurality of different types of lateral flow test kits and storing them in the database, wherein the kit templates include the specific continuous curve for color intensity indicator values associated with the corresponding analyte concentrations for the lateral flow test kits and images of the lateral flow test kit; and
obtaining the type-related information of the lateral flow test kit from the test result image, and using the stored kit template to locate the T-line region if the corresponding kit template is stored in the memory unit, wherein the XY offset of the T-line region relative to the edge of the kit is used to position the T-line region;
calculating the color intensity index value in the T-line region from the maximum value of the mean line.

14. The computer device according to claim 12, the computer program, when executed on the processor, further causing the processor to perform the following steps to determine the T-line region if no kit template corresponding to the obtained kit-type is stored in the memory:
placing the image of the lateral flow test kit vertically, and using artificial intelligence (AI) to detect the sampling well and the result area, wherein the result area includes the C-line region and the T-line region;
using color filters and grayscale filters to locate the C-line region with specific color intensities;
rotating the image into a lateral orientation such that the C-line region is perpendicular to the longitudinal axis of the lateral flow test kit;
determining whether the result area is located to the left of the sampling well;
if the result area is not located to the left of the sampling well, horizontally rotating the image so that the result area is located to the left of the sampling well;
calculating the position of the C-line region;
cropping the result area according to the ratio of the pre-determined height and width of the C-line region;
removing gradient shading of the result area using an unshading algorithm;
dividing the result area into two areas, wherein the C-line region and the T-line region are respectively located in each of the two areas.

15. The computer device according to claim 14, the computer program, when executed on said processor, further causing said processor to perform the steps of:
calculating the color intensity index values of the C-line region and the T-line region located in the two areas separately.

16. A computer-readable storage medium having stored thereon a computer program stored in a memory unit of a computer device according to claim 12, wherein the computing device performs the recited steps when said computer program is run on the computing device.
